(19) United States
(12) Patent Application Publication
(10) Pub. No.: US 2010/0312031 A1
(43) Pub. Date: Dec. 9, 2010

(54) METHOD FOR OLIGOMERIZING ALKENES

(75) Inventor: Thomas Heidemann, Viernheim (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/865,272

(22) PCT Filed: Jan. 28, 2009
(86) PCT No.: PCT/EP09/50949

§ 371 Date: Jul. 29, 2010

(30) Foreign Application Priority Data
Jan. 29, 2008 (EP) 08150795.6

(51) Int. Cl. C07C 2/24

(52) U.S. Cl. 585/326

(57) ABSTRACT

The present invention relates to a process for the oligomerization of alkenes, in which an alkene-comprising feed is provided and is subjected to an oligomerization in two successive reaction zones.

The present invention relates to a process for the oligomerization of alkenes, in which an alkene-comprising feed is provided and is subjected to an oligomerization in two successive reaction zones.

Hydrocarbon mixtures comprising short-chain alkenes, e.g. alkenes having from 2 to 6 carbon atoms, are available on an industrial scale. Thus, for example, a hydrocarbon mixture referred to as C₄ fraction which has a high total olefin content and comprises essentially alkenes having 4 carbon atoms is obtained in the processing of petroleum by steam cracking or fluid catalytic cracking (FCC). Such C₄ fractions, i.e. mixtures of isomeric butenes and butanes, are very well suited, optionally after prior removal of the isobutene and hydrogenation of the butadiene comprised, to the preparation of oligomers, in particular octenes and dodecenes. The octenes and dodecenes can be converted by hydroformylation and subsequent hydrogenation into the corresponding alcohols which are used, for example, for the preparation of plasticizers or surfactant alcohols.

For use as plasticizer alcohol, the degree of branching is critical to the properties of the plasticizer. The degree of branching is described by the iso index which indicates the average number of methyl branches in the respective fraction. Thus, for example, n-octenes make a contribution of 0, methylheptenes make a contribution of 1 and dimethylhexenes make a contribution of 2 to the ISO index of a C₈ fraction. The lower the iso index, the more linear the molecules in the respective fraction. The higher the linearity, i.e. the lower the iso index, the higher the yields in the hydroformylation and the better the properties of the plasticizer prepared therewith. In the case of phthalate plasticizers, for example, a lower iso index leads to reduced volatility and, in the case of plasticized PVC grades comprising these plasticizers, to improved cold crack behavior.

It is known that both homogeneous and heterogeneous catalysts comprising nickel or other catalytically active metals such as ruthenium, palladium, copper, cobalt, iron, chromium or titanium as active components can be used for preparing oligomers having a low degree of branching from lower olefins. However, only nickel-comprising catalysts have attained industrial importance. Homogeneous catalysts have the disadvantage compared to heterogeneous catalysts that the catalyst has to be separated off from the discharge from the reactor in an additional step. In addition, the catalyst costs per metric ton of product in the homogeneous mode of operation are generally significantly higher than in the heterogeneous mode of operation. However, when heterogeneous catalysts are used industrially, it is important to achieve very long catalyst operating lives in order to keep production downtimes as are associated with catalyst regeneration and/or catalyst replacement as few as possible.

In Catalysis Today 1990, 6, pages 329 to 349, C. T. O'Connor and M. Kojima give an overview of homogeneous and heterogeneous catalyst systems for the oligomerization of alkenes and also described, inter alia, nickel- and sulfur-comprising heterogeneous catalysts.

U.S. Pat. No. 5,113,034 describes a process for the dimerization of C₃- or C₄-olefins over a catalyst having a sulfate or tungstate as anion. Due to the high activity of the support material used, strongly branched oligomers are obtained when using these catalysts, as is also the case when using other known catalysts, e.g. catalysts based on zeolites.

The use of nickel- and sulfur-comprising catalysts for the oligomerization of alkenes is known. Heterogeneous catalysts comprising sulfur and nickel are described, for example, in FR-A-2641477, EP-A-272970, WO 95/14647, WO 01/37989, U.S. Pat. No. 2,794,842, U.S. Pat. No. 3,959,400, U.S. Pat. No. 4,511,750 and U.S. Pat. No. 5,883,036.

Various processes are known for achieving a high selectivity to essentially unbranched or slightly branched oligomers combined with very long catalyst operating lives.

WO 99/25668 describes a process for preparing essentially unbranched octenes and dodecenes by oligomerization of hydrocarbon streams comprising 1-butene and/or 2-butene and butane over a nickel-comprising heterogeneous catalyst, in which such amounts of the butane and unreacted butene which have been separated off from the reaction mixture are recirculated to the oligomerization reaction that the maximum content of oligomers in the reacted reaction mixture does not exceed 25% at any place in the reactor or reactors.

WO 00/53546 describes a process for the oligomerization of C₆-olefins over a nickel-comprising fixed-bed catalyst, in which the reaction is carried out so that the conversion into oligomerized C₆-olefins is not more than 30% by weight, based on the reaction mixture.

WO 01/72670 proposes an oligomerization process in which the discharge from the reactor is divided into two substreams and only one of the substreams is subjected to a work-up to obtain the oligomerization product and the other is recirculated directly to the oligomerization reaction.

EP 1 457 475 A2 describes a process for preparing oligomers of alkenes having from 4 to 8 carbon atoms over a nickel-comprising, heterogeneous catalyst in at least 2 successive adiabatically operated reactors.

WO 2006/111415 describes a process for the oligomerization of olefins having from 2 to 6 carbon atoms, in which an olefin-comprising feed is reacted to partial conversion in the presence of a nickel-comprising heterogeneous catalyst, the discharge is separated into a first substream and a second substream, the first substream is subjected to a work-up to obtain a fraction consisting essentially of the oligomerization product and the second substream is recirculated to the oligomerization.

There continues to be a need for an oligomerization process which makes it possible to oligomerize alkene-comprising feeds with a very high alkene conversion but without the degree of branching of the oligomers obtained being increased significantly. To achieve a very high olefin conversion, it is necessary, due to the increasing depletion of the alkene stream in the direction of the reaction coordinate, to provide a larger total catalyst volume for the oligomerization reaction or to provide an increased reaction temperature and/or a more active catalyst in the part of the catalyst bed on the outlet side. Here, an increase in the catalyst volume is economically disadvantageous for the process because of the costs associated therewith. An increase in the temperature and/or the use of a more active catalyst generally lead(s) to an unacceptable product quality of the oligomer mixture owing to the increase in the degree of branching associated therewith.

WO 2004/005224 describes a process for the oligomerization of an alkene stream in two or more successive catalyst zones using a catalyst having a molar ratio of sulfur to nickel of less than 0.5 in the first catalyst zone and using a catalyst having a molar ratio of sulfur to nickel of 0.5 or more in the last catalyst zone. This process, too, does not yet lead to a completely satisfactory conversion of the alkene comprised in the hydrocarbon feed mixture.

It has now surprisingly been found that a further increase in the conversion in the oligomerization of alkenes and a product quality which is satisfactory in terms of the degree of branching can be achieved when the oligomerization is carried out using at least two catalysts which differ in respect of their nickel content.

The invention accordingly provides a process for the oligomerization of alkenes, in which an alkene-comprising feed is provided and is subjected to an oligomerization in two successive reaction zones, wherein the reaction in the first reaction zone is carried out in the presence of a nickel-comprising heterogeneous catalyst and the reaction in the second reaction zone is carried out in the presence of a nickel-free heterogeneous catalyst.

For the purposes of the present invention, the term “oligomers” comprises dimers, trimers and higher products from the buildup reaction of the alkenes used. The oligomers are preferably essentially dimers and/or trimers. The oligomers themselves are olefinically unsaturated. Appropriate choice of the oligomerization catalysts used in the first and second reaction zones as described below makes it possible to obtain oligomers having a low degree of branching in very high yields.

The statement that the oligomerization is carried out in two “successive” reaction zones merely means, for the purposes of the invention, that the alkene-comprising feed is, viewed in the flow direction, brought into contact firstly with the nickel-comprising heterogeneous catalyst in the first reaction zone and then with the nickel-free heterogeneous catalyst in the second reaction zone. Further zones comprising catalytically active and/or inert material can be located upstream of the first reaction zone, between the first and second reaction zones and also downstream of the second reaction zone.

The sum of the volumes of the nickel-comprising heterogeneous catalyst in the first reaction zone and the nickel-free heterogeneous catalyst in the second reaction zone is preferably from 50% to 100%, particularly preferably from 75% to 100%, in particular from 90% to 100%, especially from 95% to 100%, of the total catalyst volume. In a specific embodiment, the sum of the volumes of the nickel-comprising heterogeneous catalyst in the first reaction zone and the nickel-free heterogeneous catalyst in the second reaction zone is 100%.

The volume ratio of the catalyst in the first reaction zone to catalyst in the second reaction zone is preferably in the range from 1:1 to 20:1, particularly preferably in the range from 5:1 to 10:1.

To carry out the process of the invention for the oligomerization of alkenes, it is possible to use one reactor or a plurality of (e.g. 2, 3, 4, 5, etc.) identical or different reactors. In the simplest case, a single reactor is used. If a plurality of reactors is used, these can have identical or different mixing characteristics. The individual reactors can, if desired, be divided into two or more sections by internals. Two or more reactors can be connected with one another in any way, e.g. in parallel or in series. In a preferred embodiment, two, three or four reactors connected in series are used.

The totality of the catalyst with which the alkene-comprising feed or (e.g. if the feed is introduced at two or more different points) part thereof comes into contact is also referred to as fixed catalyst bed for the purposes of the present invention. If a reactor cascade is used, the fixed catalyst bed is generally distributed over all reactors of the cascade.

A reaction zone is a section of the fixed catalyst bed in the flow direction of the feed. According to the invention, the fixed catalyst bed has a first reaction zone comprising at least one nickel-comprising heterogeneous catalyst and, downstream thereof, a second reaction zone comprising at least one nickel-free heterogeneous catalyst. The total fixed catalyst bed can consist entirely of these two reaction zones or have further reaction zones. These include, for example, upstream, intermediate or downstream reaction zones which each have a catalyst different from that/those in the adjacent first and/or second reaction zone.

A nickel-comprising catalyst is used in the first reaction zone. The first reaction zone can also comprise two or more nickel-comprising catalysts which can be present in the form of defined subzones, as a mixture or in the form of a gradient. A nickel-free catalyst is used in the second reaction zone. The second reaction zone can also comprise two or more nickel-free catalysts which can be present in the form of defined subzones, as a mixture or in the form of a gradient.

A reaction zone can be located within a part of a reactor, within a single reactor or within two or more reactors. In a preferred embodiment, the catalysts of the first and second reaction zones are each located in a single reactor or in a cascade of reactors.

The alkene-comprising feed can be fed into the fixed catalyst bed at a single point. It can also be divided up and the resulting substreams can be fed to the fixed catalyst bed at different points. When a reactor cascade is used, the substreams can be fed in, for example, at points which are located between the individual reactors. It is also possible for a substream of the alkene-comprising feed to be fed in before the beginning of a catalyst zone or (particularly when a catalyst zone extends from one reactor to the next reactor of a cascade) at the resulting point of division of the catalyst zone between the two reactors.

The process of the invention is preferably carried out continuously. Here, for example, an alkene-comprising feed is fed into the (first) reactor. This feed can comprise not only fresh alkene but also, if desired, a recycle stream from the discharge from the oligomerization reaction or from the work-up of the discharge from the reaction. If, as described in more detail below, a discharge stream is taken from the first reaction zone and subjected to a work-up to give a fraction enriched in oligomerization product and a fraction depleted in oligomerization product, the fraction depleted in oligomerization product can be at least partly recirculated to the first reaction zone. The recycle stream consists essentially of unreacted alkenes and saturated hydrocarbons. In addition, the recycle stream optionally also comprises proportions of the oligomers formed. The alkene conversion in the first reaction zone and the second reaction zone or the oligomer content in the discharge from the first reaction zone and the second reaction zone can (apart from further operating parameters such as the catalyst used, the pressure and the temperature in the reaction zones and the residence time) be controlled via the ratio of fresh alkene fed in to recycle stream.

Suitable pressure-rated reaction apparatuses for the oligomerization are known to those skilled in the art. They include the generally customary reactors for gas-solid and gas-liquid reactions, e.g. tube reactors, stirred vessels, gas recycle reactors, bubble columns, etc., which is optionally divided by means of internals. Preference is given to using tube reactors or shell-and-tube reactors.

The temperature in the oligomerization reaction is generally in the range from about 10 to 280° C., preferably from 20 to 200° C., in particular from 30 bis 190° C. and especially from 40 to 130° C. If a plurality of reactors is used, these can have identical or different temperatures. Likewise, a reactor can have a plurality of reaction regions operated at different temperatures. Thus, for example, a second reaction region of an individual reactor can be set to a higher temperature than that in the first reaction region or the second reactor of a reactor cascade can be set to a higher temperature than that in the first reactor, e.g. to achieve conversion as complete as possible.

Owing to the catalysts used according to the invention, a significantly increased temperature in the second reaction zone compared to the first reaction zone can be dispensed with. If the two reaction zones are each operated at only one temperature, the temperature in the second reaction zone is preferably not more than 30° C. higher, particularly preferably not more than 20° C. higher, in particular not more than 10° C. higher, than the temperature in the first reaction zone. If a reaction zone is operated at different temperatures or the two reaction zones are each operated at different temperatures, a temperature averaged over the volume of the zone can be determined for this/these reaction zone(s). The average temperature is determined by measuring the temperature at a sufficient number of measurement points (e.g. 3, 4, etc.) in the respective reaction zone and subsequently forming the average. The (average) temperature in the second reaction zone is then preferably not more than 30° C. higher, particularly preferably not more than 20° C. higher, in particular not more than 10° C. higher, than the (average) temperature in the first reaction zone. Owing to the catalysts used according to the invention, it is frequently possible to operate the second reaction zone at approximately the same (average) temperature or a lower (average) temperature as/than the first reaction zone.

The pressure in the oligomerization is generally in the range from about 1 to 300 bar, preferably from 5 to 100 bar and in particular from 10 to 50 bar. When a plurality of reactors is used, the reaction pressure can be different in the individual reactors.

In a specific embodiment, the temperatures and pressures used in the oligomerization are selected so that the olefin-comprising starting material is present in the liquid state or in the supercritical state.

The reaction in the first and second reaction zones is preferably carried out adiabatically. For the purposes of the present invention, this term is used in the industrial and not the physicochemical sense. Thus, the oligomerization reaction generally proceeds exothermically so that the reaction mixture experiences a temperature increase on flowing through the fixed catalyst bed. For the purposes of the present invention, adiabatic reaction conditions refers to a procedure in which the heat liberated in an exothermic reaction is taken up by the reaction mixture in the reactor and no cooling by means of cooling facilities is employed. Thus, the heat of reaction is discharged from the reactor with the reaction mixture, apart from a residual amount which is given off from the reactor to the environment by natural thermal conduction and radiation of heat. In contrast, under isothermal reaction conditions or in isothermal operation, the heat evolved in an exothermic reaction is removed by means of cooling or thermostatting facilities so that the temperature in the reactor is kept essentially constant, i.e. isothermal. In the industrial realization of these reaction conditions, too, the theoretical ideal case cannot be realized completely. Thus, it will be practically impossible to avoid a small, although in the ideal case negligibly small, part of the heat of reaction being discharged with the reaction mixture.

In a specific embodiment, part of the heat of reaction is taken from the reaction mixture during passage through the first reaction zone and/or after exit from the first reaction zone and before entry into the second reaction zone and/or during passage through the second reaction zone. A customary heat exchanger can be used for this purpose.

The reaction product produced in the first reaction zone can, in a first embodiment, be fed to the second reaction zone without the oligomers having been separated off. In this embodiment, the oligomerization product is not separated off either during passage through the first reaction zone or from the discharge from the first reaction zone.

In a second preferred embodiment, a discharge stream is taken from the first reaction zone, subjected to a work-up to give a fraction enriched in oligomerization product and a fraction depleted in oligomerization product and the fraction depleted in oligomerization product is at least partly recirculated to the first reaction zone and/or the second reaction zone.

The discharge stream can be the total reaction mixture or a substream thereof. The discharge stream can be taken off during passage through the first reaction zone or from the discharge from the first reaction zone. In a specific embodiment, the discharge stream is taken from the discharge from the first reaction zone.

In the second embodiment, preference is given to the total reaction mixture being subjected to a work-up to give a fraction enriched in oligomerization product and a fraction depleted in oligomerization product during passage through the first reaction zone or after leaving the first reaction zone.

In particular, the entire discharge from the first reaction zone is subjected to a work-up to give a fraction enriched in oligomerization product and a fraction depleted in oligomerization product.

For this purpose, the first reaction zone can, for example, be formed by two reactors connected in series, with the discharge from the first reactor or the second reactor being subjected to a work-up to give a fraction enriched in oligomerization product and a fraction depleted in oligomerization product. The fraction depleted in oligomerization product can be fed in its entirety to the next reactor in the downstream direction. It can also be fed partly to a reactor located upstream of the point at which the discharge stream has been taken off and partly to the next reactor in the downstream direction.

The first reaction zone can also be formed by, for example, three reactors connected in series, with the discharge from the first reactor or the second reactor or the third reactor being subjected to a work-up to give a fraction enriched in oligomerization product and a fraction depleted in oligomerization product. The fraction depleted in oligomerization product can once again be fed in its entirety to the next reactor in the downstream direction. It can also be fed partly to a reactor located upstream of the point at which the discharge stream is taken off and partly to the next reactor in the downstream direction.

The fractionation of the discharge stream to give a fraction enriched in the oligomerization product and a fraction depleted in the oligomerization product can be effected by customary methods known to those skilled in the art. Preference is given to fractional distillation.

The fraction enriched in the oligomerization product can, if it is not recirculated to the oligomerization, be processed further together with the oligomerization product from the second reaction zone or separately therefrom.

The fraction depleted in the oligomerization product is, in a specific embodiment, fed in its entirety to the second reaction zone.

As regards the way in which the oligomerization reaction is carried out, the disclosure of WO 99/25668, WO 01/72670, EP 1 457 475 A2 and WO 2006/111415 A1 is hereby fully incorporated by reference.

Suitable nickel-comprising heterogeneous catalysts for the first reaction zone are generally known to those skilled in the art and have been mentioned above. Preference is given to using catalysts which are known to bring about a low degree of oligomer branching. These include the catalysts described in Catalysis Today, 6, 329 (1990), in particular pages 336-338, and in DE-A-43 39 713 (=WO-A 95/14647) and DE-A-199 57 173 (=WO 01/37989), which are hereby expressly incorporated by reference.

The heterogeneous nickel-comprising catalysts used can have different structures. Both all-active catalysts and supported catalysts are suitable in principle. The latter are preferably used. The support materials can be, for example, silica, alumina, aluminosilicates, aluminosilicates having sheet structures and zeolites such as mordenite, faujasite, zeolite X, zeolite Y and ZSM-5, zirconium oxide which has been treated with acids or sulfated titanium dioxide. Precipitated catalysts which can be obtained by mixing aqueous solutions of nickel salts and silicates, e.g. sodium silicate with nickel nitrate, and optionally aluminum salts such as aluminum nitrate and calcining the precipitate are particularly useful. It is also possible to use catalysts which are obtained by incorporation of Ni²⁺ ions by ion exchange into natural or synthetic sheet silicates such as montmorillonites. Suitable catalysts can also be obtained by impregnation of silica, alumina or aluminosilicates with aqueous solutions of soluble nickel salts such as nickel nitrate, nickel sulfate or nickel chloride and subsequent calcination.

For use in the first reaction zone, preference is given to catalysts which have a molar ratio of sulfur to nickel of 0 to 0.5:1. Sulfur-free catalysts and sulfur-comprising catalysts as are described in WO 2004/005224 for use in the first catalyst zone are thus suitable. The reaction in the first reaction zone is preferably carried out in the presence of a nickel-comprising heterogeneous catalyst which has a molar ratio of sulfur to nickel of not more than 0.4:1.

Catalysts comprising nickel oxide are preferred for use in the first reaction zone. Particular preference is given to catalysts which consist essentially of NiO, SiO₂, TiO₂ and/or ZrO₂ and optionally Al₂O₃. Such catalysts are particularly preferred when the process of the invention is employed for the oligomerization of butenes. They lead to preferential dimerization over the formation of higher oligomers and give predominantly linear products. A catalyst comprising from 10 to 70% by weight of nickel oxide, from 5 to 30% by weight of titanium dioxide and/or zirconium dioxide, from 0 to 20% by weight of aluminum oxide as significant active constituents and silicon dioxide as balance is most preferred. Such a catalyst can be obtained by precipitation of the catalyst composition at pH 5 to 9 by addition of an aqueous solution comprising nickel nitrate to an alkali metal water glass solution comprising titanium dioxide and/or zirconium dioxide, filtration, drying and heating at from 350 to 650° C. For the preparation of these catalysts, reference is made specifically to DE-43 39 713 (WO 95/14647). The disclosure of this document and the prior art cited therein are fully incorporated by reference.

Catalysts which comprise nickel and sulfur and have a molar ratio of sulfur to nickel of from 0.25:1 to 0.38:1 are also preferred for use in the first reaction zone. Such catalysts are described in DE-A-199 57 173 (=WO 01/37989). They can be obtained by treating aluminum oxide with a nickel compound and a sulfur compound, either simultaneously or firstly with the nickel compound and then with the sulfur compound, with the catalyst obtained in this way subsequently being dried and calcined.

According to the invention, a nickel-free heterogeneous catalyst is used for the second reaction zone. For the purposes of the invention, a nickel-free catalyst is a catalyst which does not comprise any nickel apart from unavoidable contamination. Such catalysts generally have a nickel content of not more than 0.01% by weight, particularly preferably not more than 0.001% by weight, based on the total weight of the catalyst.

Preference is given to using a catalyst comprising aluminum oxide as support in the second reaction zone. The support material is preferably selected from among gamma-, eta- and theta-aluminum oxide and mixtures thereof. Particular preference is given to using gamma-aluminum oxide as support material.

The catalysts used in the second reaction zone preferably comprise from 1 to 15% by weight, based on the total weight of the catalyst, of sulfur in oxidic form.

To prepare such a catalyst, a support material can, for example, be brought into contact with H₂SO₄, dried and subsequently calcined.

The catalysts used in the first and second reaction zones are preferably present in particulate form. The catalyst particles generally have an average of the (greatest) diameter of from 1 to 40 mm, preferably from 2 to 30 mm, in particular from 3 to 20 mm. The catalysts include, for example, catalysts in the form of pellets, e.g. pellets having a diameter of from 2 to 6 mm and a height of from 3 to 5 mm, rings having, for example, an external diameter of from 5 to 7 mm, a height of from 2 to 5 mm and a hole diameter of from 2 to 3 mm and extrudates having various lengths and a diameter of, for example, from 1.5 to 5 mm. Such shapes are obtained in a manner known per se by tableting or extrusion on a ram extruder or screw extruder. For this purpose, customary auxiliaries, e.g. lubricants such as graphite or fatty acids (e.g. stearic acid) and/or shaping aids and reinforcing materials such as fibers comprising glass, asbestos, silicon carbide or potassium titanate, can be added to the catalyst or a precursor thereof.

Suitable alkene starting materials for the process of the invention are in principle all compounds which comprise from 2 to 6 carbon atoms and at least one ethylenically unsaturated double bond. Preference is given to alkene starting materials comprising alkenes having from 4 to 6 carbon atoms. The alkenes used for the oligomerization are preferably selected from among linear (straight-chain) alkenes and alkene mixtures comprising at least one linear alkene. These include ethene, propene, 1-butene, 2-butene, 1-pentene, 2-pentene, 1-hexene, 2-hexene, 3-hexene and mixtures thereof. Preference is given to linear α-olefins and olefin mixtures comprising at least one linear α-olefin. Particular preference is given to 1-butene, 1-pentene, 1-hexene, mixtures thereof and hydrocarbon mixtures comprising at least one such alkene. Preference is given to using an industrially available olefin-comprising hydrocarbon mixture for the oligomerization.

Preferred industrially available olefin mixtures result from hydrocarbon cracking in petroleum processing, for example by catalytic cracking such as fluid catalytic cracking (FCC), thermal cracking or hydrocracking with subsequent dehydrogenation. One suitable industrial olefin mixture is the C₄ fraction. C₄ fractions can be obtained, for example, by fluid catalytic cracking or steam cracking of gas oil or by steam cracking of naphtha. Depending on the composition of the C₄ fraction, a distinction is made between the total C₄ fraction (crude C₄ fraction), the raffinate I obtained after removal of 1,3-butadiene and the raffinate II obtained after isobutene has been separated off. A further suitable industrial olefin mixture is the C₅ fraction which can be obtained in naphtha cracking. Olefin-comprising hydrocarbon mixtures which have from 4 to 6 carbon atoms and are suitable for use in step a) can also be obtained by catalytic dehydrogenation of suitable industrially available paraffin mixtures. Thus, for example, C₄-olefin mixtures can be produced from liquefied petroleum gas (LPG) and liquefied natural gas (LNG). The latter comprises not only the LPG fraction but also relatively large amounts of relatively high molecular weight hydrocarbons (light naphtha) and are thus also suitable for preparing C₅- and C₆-olefin mixtures. The preparation of olefin-comprising hydrocarbon mixtures comprising monoolefins having from 4 to 6 carbon atoms from LPG or LNG streams can be carried out by customary processes known to those skilled in the art which generally comprise not only dehydrogenation but also one or more work-up steps. These include, for example, the removal of at least part of the saturated hydrocarbons comprised in the abovementioned olefin feed mixtures. These can, for example, be reused for preparing olefin starting materials by cracking and/or dehydrogenation. However, the olefins used in the process of the invention can also comprise a proportion of saturated hydrocarbons which are inert under the oligomerization conditions according to the invention. The proportion of these saturated components is generally not more than 60% by weight, preferably not more than 40% by weight, particularly preferably not more than 20% by weight, based on the total amount of olefins and saturated hydrocarbons comprised in the hydrocarbon starting material.

A raffinate II suitable for use in the process of the invention, has, for example, the following composition:

from 0.5 to 5% by weight of isobutane,
from 5 to 20% by weight of n-butane,
from 20 to 40% by weight of trans-2-butene,
from 10 to 20% by weight of cis-2-butene,
from 25 to 55% by weight of 1-butene,
from 0.5 to 5% by weight of isobutene
and also trace gases such as 1,3-butadiene, propene, propane, cyclopropane, propadiene, methylcyclopropane, vinylacetylene, pentenes, pentanes, etc., in amounts of not more than 1% by weight in each case.

A suitable raffinate II has the following typical composition:

| | |
|---|---|
| i-, n-butane | 26% by weight |
| i-butene | 1% by weight |
| l-butene | 26% by weight |
| trans-2-butene | 31% by weight |
| cis-2-butene | 16% by weight |

If diolefins or alkynes are present in the olefin-rich hydrocarbon mixture, these can be separated off therefrom to a concentration of preferably less than 10 ppm by weight before the oligomerization. They are preferably removed by selective hydrogenation, e.g. as described in EP-81 041 and DE-15 68 542, particularly preferably by selective hydrogenation to a residual content below 5 ppm by weight, in particular 1 ppm by weight.

In addition, oxygen-comprising compounds such as alcohols, aldehydes, ketones or ethers are advantageously substantially removed from the olefin-rich hydrocarbon mixture. For this purpose, the olefin-rich hydrocarbon mixture can advantageously be passed over an adsorbent such as a molecular sieve, in particular a molecular sieve having a pore diameter of from >4 Å to 5 Å. The concentration of oxygen-comprising, sulfur-comprising, nitrogen-comprising and halogen-comprising compounds in the olefin-rich hydrocarbon mixture is preferably less than 1 ppm by weight, in particular less than 0.5 ppm by weight.

The process of the invention is preferably carried out so that from 75 to 99%, preferably from 85 to 99%, especially from 90 to 98%, of the alkenes comprised in the alkene-comprising feed are reacted in the first reactor zone.

The process of the invention is preferably carried out so that from 30 to 99%, preferably from 50 to 99%, especially from 70 to 98%, of the alkenes comprised in the discharge from the first reaction zone are reacted in the second reactor zone.

After leaving the reactor, the oligomers formed are separated in a manner known per se from the unreacted hydrocarbons and, if desired, recirculated to the process (cf., for example, WO-A 95/14647). The fractionation is generally effected by fractional distillation.

The process of the invention differs from the known processes of this type in that it leads to a high alkene conversion combined with a low degree of branching of the oligomers which can be obtained in this way. This effect has hitherto been able to be achieved generally only by increasing the temperature in the later part of the catalyst bed or by using a more active catalyst in this region or by an increased total volume of catalyst because of the decreasing alkene content of the feed stream in the direction of the reactor outlet.

The invention is illustrated by the following, nonlimiting examples.

### EXAMPLES

### 1.) Preparation of the Catalysts

### Example 1a

### Nickel-Comprising Catalyst

A catalyst having the composition 50% by weight of NiO, 37% by weight of SiO₂ and 13% by weight of TiO₂ is prepared by the preparative method of Example 1 of DE 43 39 713 A1. The catalyst powder is mixed with 3% by weight of graphite and pressed to form 3×3 mm pellets.

### Example 1b

### Nickel- and Sulfur-Comprising Comparative Catalyst (S:Ni Ratio=1.1)

A catalyst having a nickel content of 7.9% by weight and a sulfur content of 4.32% by weight, in each case based on the total weight of the catalyst, on a γ-aluminum oxide support is prepared by the method of Example 1c of WO 2004/005224. The molar ratio of sulfur to nickel is 1.

### Example 1c

### Nickel-Free Catalyst

γ-Aluminum oxide of the type “D10-21” from BASF Aktiengesellschaft (2.3 mm extrudates, BET surface area: 210 m²/g, water absorption capacity: 0.77 ml/g, loss on ignition: 0.8% by weight) was used as support. In an impregnation drum, 28 kg of the support was sprayed at room temperature with a solution of 5.6 kg of 96% strength sulfuric acid in water (volume corresponding to the water absorption of the support) while stirring. After stirring for another 30 minutes, the support which had been impregnated in this way was dried at 120° C. for 2 hours and subsequently calcined in air at 550° C. for 5 hours. The catalyst obtained in this way comprises 5.5% by weight of sulfur in oxidic form.

### 2.) Oligomerizations

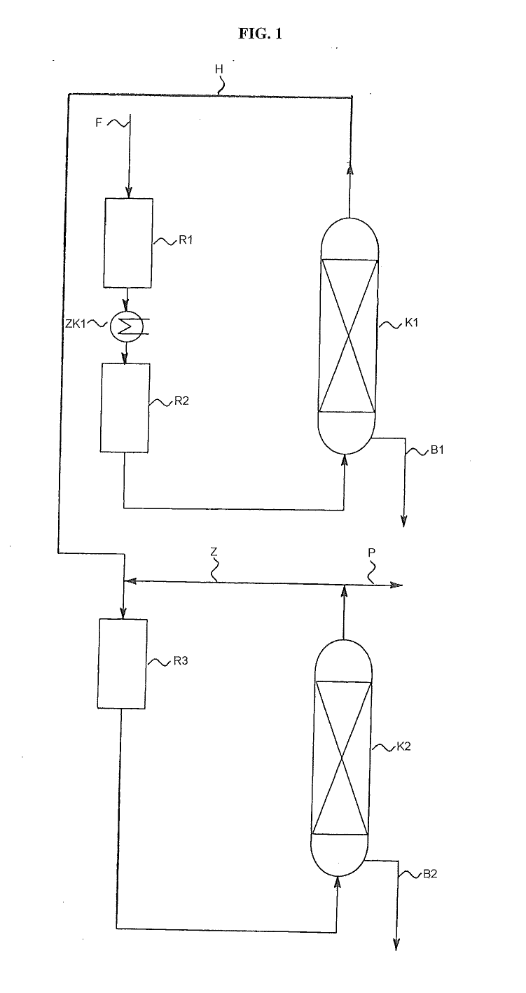

FIG. 1 shows the flow diagram of an apparatus in which the process of the invention is carried out continuously at 30 bar. All reactors R1 to R3 are operated adiabatically and each have a length of 4 m and a diameter of 0.8 m. The alkene-comprising stream (feed) is fed via the line (F) to the first oligomerization reactor (R1). The discharge from (R1) is fed via an intermediate cooling facility (ZK1) to the reactor (R2). The discharge from reactor (R2) is fractionally distilled in the column (K1) and the oligomeric reaction product is taken off as bottoms via line (B1). The overhead product (H) from the column (K1) is fed to the third oligomerization reactor (R3). The discharge from reactor (R3) is fractionally distilled in the column (K2) and the oligomeric reaction product is taken off as bottoms via line (B2). Part of the overhead stream from the column K2 is recirculated via the line (Z) to the reactor (R3) and the remaining part of the overhead stream is discharged from the apparatus via the line (P) (purge stream).

In the reactors R1 and R2, a raffinate II stream (76.4% of butenes and 23.6% of butanes) as alkene-comprising feed is firstly subjected to an oligomerization as described in Example 5 of WO 99/25668 in the presence of catalyst 1a). 90.2% of the butenes were converted into oligomers; the C₈ selectivity was 80.4%. The ISO index of the C₈ fraction was 0.99. An alkene-depleted raffinate III stream having a butene content of 24% was obtained as overhead product of the fractional distillation. Thus, 550 g of octenes (ISO index of 0.99), 140 g of higher oligomers and 310 g of raffinate III (74 g of butenes and 236 g of butanes) were thus produced per kg of raffinate II.

The raffinate III stream obtained after the oligomers have been separated off is then used for the further reaction in the reactor R3. In the comparative example, the nickel-comprising catalyst 1b) is used in the third reaction zone R3, while the nickel-free catalyst 1c) is used in the example according to the invention. The relevant data for the oligomerization reaction and the results obtained are shown in Table 1 below:

TABLE 1

| | | Cat. 1c |
|---|---|---|
| | Cat. 1b | According to |
| | Comparison | the invention |
| Space velocity of raffinate III feed | 0.5 | 0.5 |
| (stream H) (kg/l/h) | | |
| Recycle (stream Z) (kg/l/h) | 0.25 | 0.25 |
| Average reaction temperature (° C.) | 56 | 53 |
| Space-time yield of C₈⁺ (kg/l*h) | 0.084 | 0.105 |
| Butene conversion (%) | 70 | 87 |
| C₈ selectivity (%) | 73.8 | 73.1 |
| Iso index of the C₈ fraction | 1.98 | 1.99 |
| Amount of C₈⁺ produced per kg of feed | 168 | 209 |
| (stream B2) (g/kg) | | |
| Amount of C₈ produced per kg of feed (g/kg) | 124 | 153 |
| Amount of purge produced per kg of feed | 832 | 791 |
| (stream P) (g/kg) | | |
| Butene content of purge stream P (%) | 8.7 | 3.9 |
| Amount of C₈⁺ produced per 310 g of | 52 | 65 |
| feed (g) | | |
| Amount of C₈ produced per 310 g of feed (g) | 38 | 47 |

It can clearly be seen that when, according to the invention, a nickel-comprising catalyst is used in the first reaction zone (reactors R1 and R2) and a nickel-free catalyst is used in the second reaction zone (reactor R3) under otherwise comparable conditions as regards the space velocity over the catalyst and reaction temperature, a significantly higher butene conversion can be achieved at comparable C₈ selectivity (70% vs. 87%). Thus, 588 g of octenes having an iso index of 1.05 are produced per kg of raffinate II in the comparative example using a combination of catalyst 1a) and 1b). In the case of the combination according to the invention of catalyst 1a with catalyst 1c, 597 g of octenes having an iso index of 1.06 are obtained per kg of raffinate II.

1.-11. (canceled)

12. A process for the oligomerization of alkenes which comprises providing an alkene-comprising feed and subjecting to an oligomerization in two successive reaction zones, wherein the reaction in the first reaction zone is carried out in the presence of a nickel-comprising heterogeneous catalyst and the reaction in the second reaction zone is carried out in the presence of a nickel-free heterogeneous catalyst.

13. The process according to claim 12, wherein from 75 to 99% of the alkenes, based on the alkene content of the alkene-comprising feed, are reacted in the first reaction zone.

14. The process according to claim 12, wherein from 30 to 99% of the alkenes in the discharge from the first reaction zone, based on the alkene content of the discharge from the first reaction zone, are reacted in the second reaction zone.

15. The process according to claim 12, wherein the first reaction zone is carried out in the presence of a catalyst comprising from 10 to 70% by weight of nickel oxide, from 5 to 30% by weight of titanium dioxide and/or zirconium dioxide and from 0 to 20% by weight of aluminum oxide as significant active constituents and optionally silicon dioxide as balance to 100% by weight.

16. The process according to claim 12, wherein the first reaction zone is carried out in the presence of a catalyst which can be obtained by treating aluminum oxide with a nickel compound and a sulfur compound, either simultaneously or firstly with the nickel compound and then with the sulfur compound, and subsequent drying and calcination of the catalyst obtained in this way, with the catalyst obtained having a molar ratio of sulfur to nickel of from 0.25:1 to 0.38:1.

17. The process according to claim 12, wherein the second reaction zone is carried out in the presence of a catalyst comprising aluminum oxide as support and from 1 to 15% by weight, based on the total weight of the catalyst, of sulfur in oxidic form.

18. The process according to claim 15, wherein the second reaction zone is carried out in the presence of a catalyst comprising aluminum oxide as support and from 1 to 15% by weight, based on the total weight of the catalyst, of sulfur in oxidic form.

19. The process according to claim 12, wherein the volume ratio of the catalyst in the first reaction zone to catalyst in the second reaction zone is in the range from 1:1 to 20:1.

20. The process according to claim 18, wherein the volume ratio of the catalyst in the first reaction zone to catalyst in the second reaction zone is in the range from 5:1 to 10:1.

21. The process according to claim 12, wherein the alkene-comprising feed comprises at least one olefin having from 2 to 6 carbon atoms.

22. The process according to claim 12, wherein the alkene-comprising feed comprises a mixture of butenes and butanes.

23. The process according to claim 20, wherein the alkene-comprising feed comprises a mixture of butenes and butanes.

24. The process according to claim 12, wherein the reaction product produced in the first reaction zone is fed to the second reaction zone without the oligomers having been separated off.

25. The process as claimed in claim 12, wherein a discharge stream is taken from the first reaction zone, subjected to a work-up to give a fraction enriched in oligomerization product and a fraction depleted in oligomerization product and the fraction depleted in oligomerization product is at least partly recirculated to the first reaction zone and/or the second reaction zone.
